(19) United States
(12) Patent Application Publication
(10) Pub. No.: US 2009/0325281 A1
(43) Pub. Date: Dec. 31, 2009

(54) COMPOSTER

(76) Inventors: Carl R. Stanford, Clearfield, UT (US); Kent Ashby, Logan, UT (US)

Correspondence Address: Workman Nydegger;1000 Eagle Gate Tower, 60 East South Temple, Salt Lake City, UT, 84111, US

(21) Appl. No.: 12/492,876

(22) Filed: Jun. 26, 2009

Related U.S. Application Data
(60) Provisional application No. 61/076,376, filed on Jun. 27, 2008.
(60) Provisional application No. 61/107,609, filed on Oct. 22, 2008.

(51) Int. Cl. C12M 1/00

(52) U.S. Cl. 435/290.1

(57) ABSTRACT

A composter may include a body that is sized and configured to receive composting materials. The body, which may be constructed from blow-molded plastic, may include sides that have an inner wall, an outer wall and a hollow interior portion disposed between the inner and outer walls. The inner wall may includes a plurality of inwardly extending channels, which extend towards the outer wall, and one or more outwardly extending portions that extend into the body of the composter. The inner wall, outer wall, hollow interior portion and channels are preferably integrally formed as part of a unitary, one-piece construction during the blow-molding process. The composter may also include a frame that is connected to the body, and sized and configured to support the body above a support surface. The body is preferably rotatably connected to the frame to allow the composter to freely rotate and a latch or lock may be used to secure the body in a fixed position.

### CROSS-REFERENCE TO RELATED APPLICATIONS

The present application claims priority to and the benefit of U.S. Provisional Patent Application Ser. No. 61/076,376, entitled COMPOSTER, which was filed on Jun. 27, 2008.

The present application claims priority to and the benefit of U.S. Provisional Patent Application Ser. No. 61/107,609, entitled COMPOSTER, which was filed on Oct. 22, 2008. Each of these applications is incorporated by reference in its entirety.

### BACKGROUND OF THE INVENTION

1. Field of the Invention

The present invention generally relates to composting and, in particular, to composters.

2. Description of Related Art

Composted organic material may be used for a variety of purposes. For example, many gardeners and horticulturists use composting to help grow plants such as herbs, fruits and vegetables. Composting is frequently used because it may save money on fertilizer and other store-bought gardening products. In addition, composting may be an easy, convenient way to dispose of garden refuse and may help conserve dwindling landfill space. Composting may also help restore nutrients back into the soil and help reduce erosion and nutrient run-off.

Traditionally, composting involves combining various types of organic waste, such as leaves, yard trimmings and household waste, into a composting pile. Combining various types of organic waste starts a microbial process that converts the organic waste into useful mulch and decomposed organic matter that may be used to fertilize and enhance the soil in gardens, vegetable beds and landscaped areas.

Compost piles, however, may be restricted in some areas and many neighbors may not appreciate an uncontained compost pile. In addition, many traditional compost piles may attract unwanted scavenging animals, rodents, insects and the like. Further, because compost piles require a relatively large area, compost piles may not be used in urban or metropolitan areas.

It is also known to use a compost bin to hold the organic waste scraps to be turned into compost. A compost bin may keep the compost in a smaller, contained area than a compost pile.

### BRIEF SUMMARY OF THE INVENTION

A need therefore exists for a composter that eliminates or diminishes the disadvantages and problems described above.

One aspect is a composter that may include a compartment for storing composting materials and/or compost. For example, the compartment may be filled with a desired amount of composting materials, such as one or more organic materials, and the materials may decompose into compost. The compartment may include a lid or cover, which may open and close, which may provide easy access to the compartment. The lid may be securely connected to create a water-tight seal, which may help prevent rain from entering the composter and it may help maintain the desired moisture within the composter. The lid may be used to help ensure that the compost has adequate water and that the composting materials remain moist.

Another aspect is a composter that may include a plurality of storage compartments. The multiple compartments may allow composting of different materials and composting at different times. For example, one compartment may be used to create fresh compost that may be formed in two to three weeks, while another compartment may be used to create cured compost that may be formed in two to three months. The multiple compartments may also allow different materials to be composted and the materials may be composted at different rates. For instance, a first compartment may be filled with desired amounts and types of materials, and these materials may be allowed to compost for a period of time. A second compartment may be filled at the same or different time, and it may be filled with the same or different amounts and types of materials, and these materials may be allowed to compost for a length of time. Thus, the multiple compartments may allow different materials to be composted, different types and amounts of compost to be created, different degrees of composting to be completed, etc. This may also allow the composting process to be completed sooner or at different times.

Still another aspect is a composter that may include a plurality of storage compartments and one or more indicators. The indicators may be sized and configured to selectively indicate which, if any, compartments should receive additional composting materials. The indicators may indicate, for example, that a first compartment is in the accumulation stage and more composting materials should be added. The indicators may also indicate that a second compartment is in the composting stage and no additional material should be added. In addition, the indicators may indicate both the first and second compartments are in the accumulation stage or composting stage. The indicators may also provide other information, if desired.

Yet another aspect is a composter that may include multiple storage compartments and partitions may be disposed between the storage compartments. The partitions may be used to divide the composter into storage compartments of equal or different sizes. The partitions could be movable to allow the size of the compartments to be adjusted. In addition, the partitions may be used to completely separate the compartments, or the partitions may allow some communication or interaction between the compartments. The partitions may be constructed from metal (such as steel or aluminum), plastic (such as vinyl or nylon), fabric (such as cloth or polyester), or other suitable natural or synthetic materials. The partitions may be solid structures or they may include openings, holes, apertures or the like to allow communication between the compartments.

A further aspect is a composter that may include a partition which permits fluids to pass between the storage compartments. For example, the partition may include one or more openings and fluids such as leachate, which may be a product or solution formed by leaching, may pass through the openings. The openings may be disposed proximate a lower or outer portion of the partition and the openings may be sized and configured to help control the amount of leachate passing between the compartments. The partition may also include one or more permeable sections that are sized and configured to allow leachate to pass between the compartments. For instance, the partition may be at least partially constructed from permeable sections such as screens, mesh, and the like. Because the partition may permit leachate to pass between the compartments, this may allow drier materials in one compartment to absorb leachate from another compartment. This may, for example, eliminate the need to add water, moisture or other materials to the compartment with the drier materials. This may also eliminate the need to remove water, moisture and/or leachate from the other compartment. This may expedite the composting process and/or make composting quicker and easier. In addition, because the partition may permit leachate to pass between the compartments, this may allow the leachate to be evenly distributed within the compartments.

Another further aspect is a composter that may include a movable partition, which may allow compartments of different sizes to be created. For example, the partition may be slidable between various locations to allow the size of a compartment to be increased or decreased. The partition may also be slidable to allow the size of one compartment to be increased and the size of another compartment to be decreased. In order to facilitate movement of the partition, the partition may be disposed in one or more receiving portions, along a track, on rollers, and the like. The partition may also be deformed or bendable to facilitate movement. For example, the partition may be constructed from a flexible material, such as fabric, and the partition may be distorted or at least partially collapsed to allow the partition to be moved.

Advantageously, the movable partition may allow the size of the compartments within the composter to be readily changed. For example, when the material in a first compartment decomposes, it may be reduced in size. If desired, the position of the partition may be adjusted because of the decreasing size of the decomposing materials. This may increase the size of the second compartment and permit additional material to be added to the second compartment, which may increase the efficiency of the composter.

A still further aspect is a composter that may include a rotatable body or tumbler which is supported by a frame. The frame preferably elevates the body above the ground to help prevent rodents, insects and animals from getting into the composter. The rotating body may help agitate the material in the composter, which may help the material to decompose more evenly and speed the composting process. Preferably, the body is easily rotated to allow the compost to be effortlessly mixed. The composter may include a lock or latch to secure the body in a fixed position.

The composter may allow materials to be quickly and easily turned into compost. For example, the composer may be filled with materials that have a smaller particle size and the composer may be easily turned to mix the materials. Because the smaller particles may have increased surface area, that may allow the materials to be faster and more completely composted.

Another further aspect is a composter that may include one or more elongated members, such as hollow tubes, that are at least partially disposed in the rotatable body or tumbler. In particular, the elongated members are preferably disposed within the compartments and the members may agitate the material as the composter is rotated.

Another aspect is a composter that may provide increased aeration, which may speed the composting process. In particular, the composter may provide more oxygen to the compost during the composing process. For example, the composter may include elongated members, such as hollow tubes, and one or both ends of the elongated members may extend through the exterior wall of the body. The elongated members may also include openings or passages to facilitate gas flow within the composter and/or allow outside air to be introduced into the composter. Advantageously, the elongated members may provide both increased air flow and agitation of the materials when the composter is rotated, which may expedite the decomposition process.

Still another aspect is a composter that may include a partition which may be disposed at an angle. For example, the partition may be disposed perpendicular to an inner wall of the body or an axis of rotation of the body. The partition may also be disposed at other angles such as about a 30° angle, a 45° angle, a 60° angle or the like. The partition may also be movable relative to the inner wall of the body or the axis of rotation of the body. This may allow, for example, the partition to be disposed generally perpendicular to the axis, generally aligned with the axis, spaced apart from the axis and/or generally parallel to the axis.

Yet another aspect is a composter that may include one or more components constructed from plastic. Preferably, the components are constructed from blow-molded plastic. For example, the body, lid and/or partition may be constructed from blow-molded plastic. The blow-molded plastic components may include a pair of spaced apart walls and a hollow interior portion disposed between the walls. The walls and hollow interior portion may be integrally formed as part of a unitary, one piece structure during the blow-molding process. Significantly, the spaced apart walls and hollow interior portion may help insulate the composter. The insulation may help reduce the lost of heat generated during the composting process, which might help speed the composting process. The plastic components may also allow a durable composter to be constructed because the plastic may not rust, oxidize or corrode when exposed to the elements. In addition, the blow-molded plastic components may allow a lightweight composter to be created, which may reduce shipping costs and facilitate movement of the composter. The composter may also be constructed from other durable materials, such as metal, which may create a tough and long-lasting composter.

A further aspect is a composter that may be readily portable. For instance, the composter may be quickly and easily movable between a garage, backyard, patio, shed and the like. The composter may also include an aesthetic design, which may facilitate use of the composter in different environments. For example, the composter may provide an unobtrusive addition to a yard, courtyard or garden.

A still further aspect is a composter that may be easily assembled. For example, the composter may be disposed in a first configuration for shipping, storage and/or transport and a second configuration for use. Desirably, the composter may be at least partially unassembled in the shipping configuration. In addition, two or more components of the composter may at least partially nest together in the shipping configuration, which may help reduce the size of the packaging necessary to ship the composter and that may help reduce shipping costs. The composter may also be quickly and easily disassembled for storage, if desired.

Yet another further aspect is a composter may include few parts, which may facilitate manufacturing, shipping and assembly of the composter. For example, the composter may include a frame that can be quickly and easily assembled or disassembled. The composter may also include a body or tumbler that is constructed from several components that may be rapidly connected and disconnected in a straightforward manner. The tumbler and the frame may also be quickly and easily connected and disconnected. This may allow a consumer to assemble the composter in a desired location for use and it may allow the consumer to disassemble the composter for storage.

Another aspect is a composter that may include a body with a compartment that is sized and configured to receive composting materials. The body may include a first portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction; a second portion including an inner wall, an outer wall and a hollow interior portion disposed between the inner wall and the outer wall, the inner wall including a plurality of inwardly extending channels, the inner wall, the outer wall, the hollow interior portion and the at channels being integrally formed as part of a unitary, one-piece construction; and a third portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction. The composter may also include a frame connected to the body and the frame may be sized and configured to support the body above a support surface. The first portion may form a first side of the body, the second portion may form a second side of the body and the third portion may form a third side of the body. In addition, the compartment may be at least partially defined by the first portion, the second portion and the third portion. Further, the first portion, the second portion and the third portion may form a three-sided body.

Still another aspect is a composter that may include an outer wall of the first portion of the body with an inwardly curved, generally concave portion that is disposed between a first outwardly curved, generally convex portion and a second outwardly curved, generally convex portion. The composter may also include a handle integrally formed in the outer wall of the first portion of the body as part of the unitary, one-piece construction. In addition, the composter may include a lid attached to the first portion of the body, the lid may cover an opening to the compartment, the lid may include an inner wall, an outer wall and a hollow interior portion disposed between the inner wall and the outer wall, the inner wall may include a plurality of inwardly extending channels, the inner wall, the outer wall, the hollow interior portion and the channels may be integrally formed as part of a unitary, one-piece construction.

Yet another aspect is a composter that may include a front portion connected to the first portion, the second portion and the third portion of the body, the front portion may include an inner wall, an outer wall and a hollow interior portion disposed between the inner wall and the outer wall, the inner wall may include a plurality of inwardly extending channels, the inner wall, the outer wall, the hollow interior portion and the channels may be integrally formed as part of a unitary, one-piece construction. The composter may also include a rear portion connected to the first portion, the second portion and the third portion of the body, the rear portion may include an inner wall, an outer wall and a hollow interior portion disposed between the inner wall and the outer wall, the inner wall may include a plurality of inwardly extending channels, the inner wall, the outer wall, the hollow interior portion and the channels may be integrally formed as part of a unitary, one-piece construction. In addition, the composter may include an axle extending through the front portion and the rear portion of the body, the axle may be rotatably connected to the frame. If desired, at least some of the channels in the front portion may be aligned with a central portion of the front portion and extend radially outwardly. Additionally, the composter may include one or more depressions at least partially disposed in at least some of the channels in the front portion of the body. Further, the channels in the front portion of the body may be disposed in a generally symmetric pattern that covers at least a majority of the inner wall of the front portion of the body.

Still yet another aspect is a composter that may include an inwardly extending portion on the inner wall of the first portion of the body, the inwardly extending portion may be integrally formed as part of the unitary, one-piece construction. The inwardly extending portion may have a generally X-shaped configuration and may be sized and configured to facilitate mixing of the composting materials in the body. If desired, the inwardly extending portion may have a height generally equal to or greater than an average depth of the channels in the inner wall of the first portion of the body. Additionally, the plurality of inwardly extending channels in the inner wall of the first portion of the body may include at least five generally aligned channels. The plurality of inwardly extending channels in the inner wall of the first portion of the body may also include a first set of generally aligned channels that are spaced apart from a second set of generally aligned channels. In addition, the plurality of inwardly extending channels in the inner wall of the first portion of the body may include a first set of generally aligned channels that are disposed generally perpendicular to a second set of generally aligned channels. Further, at least one depression may be disposed in at least a majority of the inwardly extending channels in the inner wall of the first portion of the body.

These and other aspects, features and advantages of the present invention will become more fully apparent from the following brief description of the drawings, the drawings, the detailed description of preferred embodiments and appended claims.

### BRIEF DESCRIPTION OF THE DRAWINGS

The appended drawings contain figures of preferred embodiments to further illustrate and clarify the above and other aspects, advantages and features of the present invention. It will be appreciated that these drawings depict only preferred embodiments of the invention and are not intended to limits its scope. Additionally, it will be appreciated that while the drawings may illustrate preferred sizes, scales, relationships and configurations of the invention, the drawings are not intended to limit the scope of the claimed invention. The invention will be described and explained with additional specificity and detail through the use of the accompanying drawings in which:

FIG. 1 is a perspective view of an exemplary composter;

FIG. 2 is an enlarged, upper perspective view of a portion of the composter shown in FIG. 1, illustrating an interior portion of the composter with the lid removed;

FIG. 3 is a perspective view of another exemplary composter;

FIG. 4 is an enlarged, upper perspective view of a portion of the composter shown in FIG. 3;

FIG. 5 is a perspective view of an exemplary indicator;

FIG. 6 is a perspective view of another exemplary indicator;

FIG. 7 is another perspective view of the indicator shown in FIG. 6;

FIG. 8 is a perspective view of still another exemplary composter, illustrating the composter in a shipping configuration; and

FIG. 9 is an exploded view of the composter shown in FIG. 8, illustrating the components in the shipping configuration.

### DETAILED DESCRIPTION OF THE PREFERRED EMBODIMENTS

The present invention is directed towards a composter, which is described in more detail below. It will be appreciated that the composter may also have other suitable shapes, sizes, configurations and arrangements depending, for example, upon the intended use of the composter. It will also be appreciated that the composter does not have to include all of the various parts, components, features, aspects and characteristics disclosed herein, and it could include other parts, components, features, aspects and characteristics.

Additionally, to assist in the description of the composter, words such as top, bottom, front, rear, right and left are used to describe the accompanying figures. It will be appreciated, however, that the present invention can be located in a variety of desired positions including various angles, sideways and even upside down. A detailed description of the composter now follows.

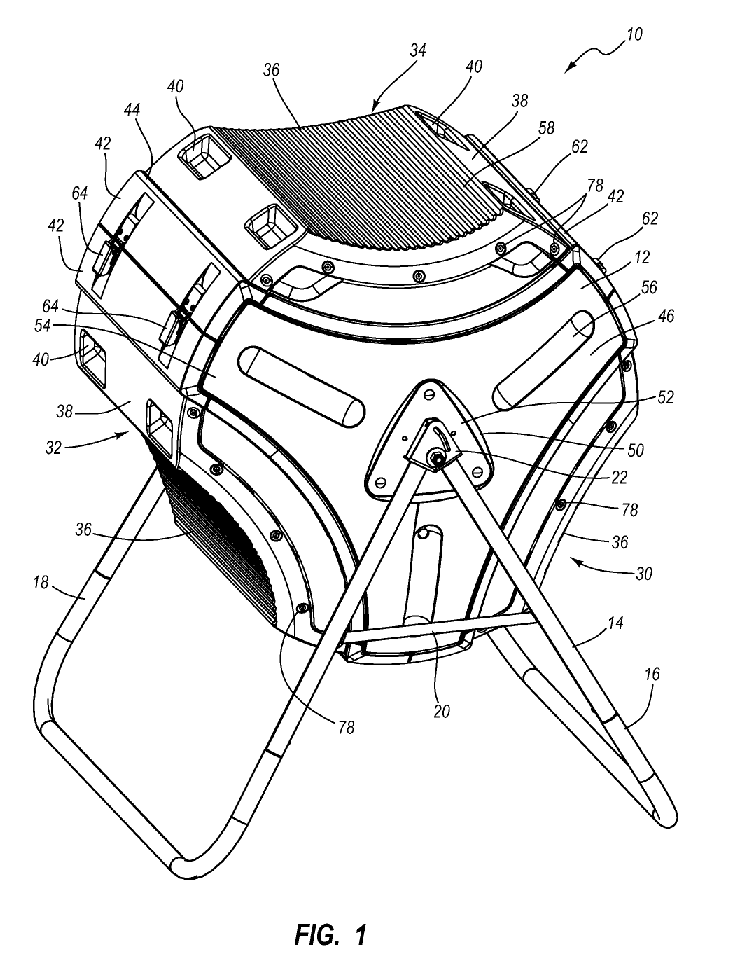

As seen in FIG. 1, an exemplary composter 10 may include a body 12 that is supported by a frame 14. The frame 14 is preferably sized and configured to support the body 12 above the ground, which may help prevent rodents, insects and animals from getting into the composter 10. In greater detail, the frame 14 may include a first support structure 16 and a second support structure 18, which may be connected by a connecting member 20 to help create a strong and sturdy base for the composter 10. As shown in the accompanying figures, the first and second support structures 16, 18 may have a generally U-shaped configuration and may be connected at two different locations to the body 12. For example, the first and second support structures 16, 18 may be connected to the body 12 by a brace 22. As discussed below, the frame 14 may be formed by one or more parts, which may facilitate shipping and manufacturing of the composter 10. It will be understood that the frame 14 may have other shapes, sizes, configurations and arrangements depending, for example, upon the size and/or intended use of the composter 10.

The body 12 is preferably rotatably connected to the frame 14 so that the body, which may also be referred to as a tumbler, can be easily turned or rotated. For example, the body 12 may be connected to the frame 14 by one or more axles, bearings, rollers or the like. As discussed in greater detail below, rotating the body 12 may help speed the composting process and allow materials to decompose more evenly. In addition, if the body 12 is easy to rotate, then the compost to be easily mixed and it may encourage turning and/or mixing of the compost on a more frequent basis such as daily or weekly.

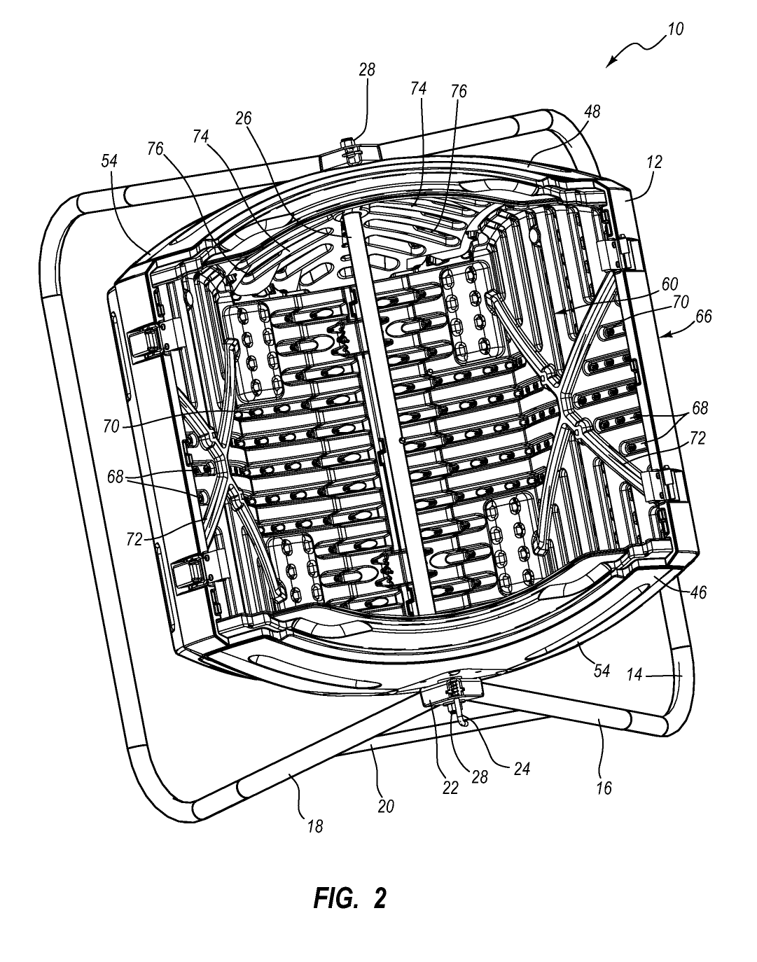

The body 12, however, may be relatively difficult to turn and/or secured in a fixed location by a latch, lock, fastener, catch, etc. For example, as shown in FIG. 2, a spring-loaded latch 24 may be used to secure the body 12 in a fixed position. This may allow the body 12 to remain in a fixed position, which may facilitate adding or removing materials from the body. In addition, if the body 12 can be secured in a fixed position, then the body may not be undesirably or inadvertently moved. Preferably special tools or other instruments are not required to turn the body 12, but they could be required if desired.

As shown in FIG. 2, the frame 14 may be connected to an axle 26 and the axle may extend through the body 12. The axle 26 may be pivotally connected to the frame 14 to facilitate rotation of the body 12. In particular, the axle 26 may be connected to the frame 14 by fasteners 28 such as nuts attached to threaded portions of the axle. It will be appreciated that the body 12 may be connected to the frame 14 using other suitable structures and devices. Further, while the body 12 is preferably rotatably connected to the frame 14, the body does not have to be rotatably connected to the frame.

The body 12 may be a three-sided structure including a first side 30, a second side 32 and a third side 34. The sides 30, 32 and 34 preferably have the same general size, shape, configuration and arrangement, which may help create an aesthetically pleasing design. The sides 30, 32 and 34, however, could have other suitable sizes, shapes, configurations and arrangements depending, for example, upon the intended use of the composter 10.

In greater detail, as best seen in FIG. 1, each of the sides 30, 32 and 34 preferably has an inwardly curved portion 36 and the inwardly curved portion may have a textured surface such as a plurality of ribs and/or grooves. Each of the sides 30, 32 and 34 may also have a first outwardly curved portion 38 disposed on each end of the inwardly curved portion 36. The first outwardly curved portions 38 may include one or more inwardly extending portions 40, which may form handles or grips. The inwardly extending portions 40 are preferably sized and configured to allow a user to help grasp and/or rotate the body 12. For example, a user may grasp one or more inwardly extending portions 40 to move the body 12 into the desired position or to rotate the body to mix and/or aerate any materials disposed within the body.

The body 12 may also include second outwardly curved portions 42 that are disposed adjacent to the first outwardly curved portions 38. The first and second outwardly curved portions 38, 42 may extend outwardly from the body 12 by different amounts or distances. For example, the second outwardly curved portion 42 may extend outwardly a greater distance than the first outwardly curved portion 38. The first and second outwardly curved portions 38, 42 may also be separated by a distance or gap 44. This gap 44 may be formed by an angled surface that connects the first and second outwardly curved portions 38, 42.

In addition, the body 12 may include a front portion 46 and a rear portion 48. As seen in FIG. 1, the front portion 46 may include a center section 50, which may be recessed and sized and configured to a plate or mounting bracket 52. The mounting bracket 52 may facilitate attachment of the body 12 to the frame 14. For example, the mounting bracket 52 may include an opening that is sized and configured to receive the axle 26. The mounting bracket 52 may also help securely support the axle 26 and the mounting bracket is preferably constructed from a strong and durable material such as metal. The mounting bracket 52 may be connected to the body 12 by one or more fasteners.

The front portion 46 may also include an outwardly extending portion 54 that extends outwardly from the body 12. As best shown in FIG. 2, the outwardly extending portion 54 may also have a curved or bowed configuration. The front portion 46 may include inwardly extending portions 56, which are preferably sized and configured to allow a user to help grasp and/or rotate the body 12. For example, a user may grasp one or more inwardly extending portions 56 to move the body 12 into the desired position or to rotate the body.

The composter 10 may also include a lid 58 that provides access to an interior portion 60 of the body 12. The lid 58 may be securely connected to the body 12 to create a water-tight seal, which may help prevent rain or water from entering the composter. The water-tight lid 58 may also help maintain a desired amount of moisture within the composter 10. For example, the lid 58 may be used to help ensure that the compost has adequate water and that the composting materials remain moist. The lid 58 may be connected to the body 12 by hinges 62 and the lid may be secured in a closed position by latches 64. If desired, the lid 58 may be removably attached to the body 12 and the lid could be attached to the body using other suitable devices and methods.

The interior portion 60 may include one or more compartments 66 for storing and/or composting materials. For example, the compartment 66 may be filled with a desired amount of composting materials, such as one or more organic materials, and the materials may decompose into compost. The lid 58 may allow the composting materials to be quickly and easily added or removed as desired. When the lid 58 is in the closed position, the body or tumbler 12 may be rotated to mix the materials, which may expedite the composting process.

The body 12 and the lid 58 are preferably constructed from strong, durable and weather resistant materials. For example, the body 12 and lid 58 may be constructed from blow-molded plastic, which may allow a durable composter 10 to be constructed because the plastic may not rust, oxidize or corrode when exposed to the elements. In addition, the blow-molded plastic components may be quickly and efficiently manufactured. Further, because the blow-molded plastic components may be lightweight, that may allow a sturdy, robust and lightweight composter 12 to be constructed. The lightweight blow-molded plastic components may also help reduce shipping costs. While the body 12 and lid 58 are preferably constructed from blow-molded plastic, it will be appreciated that these and other components of the composter 10 may be constructed from other materials and processes with suitable properties and characteristics. For example, the frame 14 may be constructed from metal, such as hollow steel tubes, and the various brackets, fasteners and the like may also be constructed from materials such as metal.

The blow-molded plastic components may include a pair of spaced apart walls and a hollow interior portion may be disposed between the walls. The walls and hollow interior portion are preferably integrally formed as part of a unitary, one piece structure during the blow-molding process. In particular, as best seen in FIGS. 1 and 2, the sides 30, 32, 34; and the front and rear portions 46, 48 of the body 12 may be constructed from blow-molded plastic. The blow-molded plastic components may include an interior surface or wall and an exterior surface or wall. As seen in FIG. 1, the exterior surfaces of the body 12 may have various shapes, sizes, configurations and arrangements. As seen in FIG. 2, the interior surfaces of the body 12 may also have various shapes, sizes, configurations and arrangements. The interior and exterior surfaces are preferably spaced apart by a distance, which may be a generally constant distance.

In greater detail, the interior and exterior surfaces may include a plurality of inwardly and outwardly extending portions. For example, the outer surface of the sides 30, 32, 34 may include the inwardly curved portions 36 and handles 40; and the outer surface of the front and rear portions 46, 48 may include the outwardly extending portions 54 and the inwardly extending portions 56.

As seen in FIG. 2, the inner surface of the sides 30, 32, 34 preferably includes a plurality of inwardly extending channels 68. In particular, the sides 30, 32, 34 may include a plurality of channels 68 that are aligned in a first direction and a plurality of channels that are aligned in a second direction. For example, as shown in the accompanying figures, the sides 30, 32, 34 may include five or seven generally aligned channels 68 in a first direction and five or seven channels generally aligned in a second direction. The channels 68 may be disposed at an angle such as about 90° degrees, but the channels could be disposed at any suitable angle.

One or more depressions or tack-offs 70 may be at least partially disposed within one or more of the channels 68. For example, a single depression 70 may be disposed within one of the channels 68 or multiple depressions may be disposed within the channels. The depressions 70 are preferably closely spaced and the number of depressions may be dependent upon the size and shape of the channels 68. For example, the larger the channel 68, the more depressions 70 may be disposed within a given channel. The depressions 70 preferably extend towards and may engage the outer surfaces of the sides 30, 32, 34. The depressions 70 may help create a strong and sturdy structure because they may help support the outer surfaces of the sides 30, 32, 34.

As shown in FIG. 2, the channels 68 and the depressions 70 are preferably disposed in a pattern, which may help create a strong and lightweight body 12. The pattern is preferably relatively symmetrical and may cover substantially all or a majority of the inner surfaces of the sides 30, 32, 34. In addition, while the sides 30, 32, 34 may have generally the same pattern, the sides could have different patterns if desired.

In greater detail, the pattern may include a first channel 68 disposed on one side and a corresponding channel may be disposed on an opposing side, and these channels may be generally aligned. In addition, one or more depressions 70 may be disposed in the channels 68. The channels 68 and depressions 70 preferably cover at least a substantial portion or majority of the inner surface of the sides 30, 32, 34.

The inner surface of the sides 30, 32, 34 may also include one or more inwardly extending portions 72. As shown in FIG. 2, the inwardly extending portions 72 may have a generally X-shaped configuration and it may be disposed between the channels 68 and/or depressions 70. Preferably, the inwardly extending portions 72 are disposed on the curved inner surfaces of the inwardly curved portions 36 of the sides 30, 32, 34. The inwardly extending portions 72 may be sized and configured to help mix the materials within the compartment 66. For example, when the tumbler 12 is rotated, the inwardly extending portions 72 may help agitate the materials in the compartment 66, which may help the materials decompose more evenly. The inwardly extending portions 72 may also allow the compartment 66 to be filled with materials that have a smaller particle size, which may allow the materials to be faster and more completely composted. In particular, the smaller-sized particles may have increased surface area and that may expedite the composting process.

The channels 68, depressions 70 and inwardly extending portions 72 are preferably integrally formed as part of a unitary, one-piece structure. In particular, the channels 68, depressions 70 and inwardly extending portions 72 are preferably integrally formed during the blow-molding process as part of the unitary, one-piece structure. For example, the sides 30, 32, 34 may each be integrally formed with the channels 68, depressions 70 and inwardly extending portions 72, and then the sides may be connected to form the body 12. One or more of the sides 30, 32, 34 may also be integrally formed as part of the unitary, one-piece structure, if desired.

The front and rear portions 46, 48 of the body 12 may also include channels 74 and depressions 76. For example, the front and rear portions 46, 48 may include a plurality of channels 74 and at least a portion of these channels may be generally aligned with a center of the front and rear portions. The channels 74 may also be configured with a first end disposed towards a center of the front or rear portion 46, 48 and a second end disposed towards an outer edge or perimeter of the front or rear portion. One or more depressions 76 may be disposed in the channels 74. For example, the smaller channels 74 may only include a single depression 76 and the larger channels may include a number of depressions. Although not shown in the accompanying figures, the front and rear portions 46, 48 of the body 12 may also include one or more inwardly extending portions, which may be similar to the inwardly extending portions 72.

The channels 74 and depressions 76 are preferably integrally formed as part of a unitary, one-piece structure with the front and rear portions 46, 48, respectively. In particular, the grooves 74 and depressions 76 are preferably integrally formed during the blow-molding process as part of the unitary, one-piece structure to form the front portion 46 and the rear portion 48.

As shown in FIG. 1, the sides 30, 32, 34 and the front portion 46 may be connected by fasteners 78. Similarly, the rear portion 48 may be connected to the sides 30, 32, 34 by fasteners. This would allow the body 12 of the composter 10 to be primarily constructed by the sides 30, 32, 34 and the front and rear portions 46, 48. These various components may be separately formed and then interconnected, or these components may be integrally formed as part of a unitary, one-piece structure. It will be appreciated that the body 12 may have any suitable number, shape, size, configuration or arrangement of components depending, for example, upon the intended use of the composter 10. Thus, the body 12 could also have four, five, six or more sides; and the body could include any number of front and rear portions.

The lid 58 preferably covers an opening in one of the sides 30, 32, 34 and the lid may be sized and shaped so that the side has the same general size, shape, configuration and arrangement as the other sides. Thus, the body 12 of the composter 10 may have a balanced or symmetrical appearance. The lid 58 may include an inner surface that is similar to the inner surface of the sides. For example, the lid 58 may include a plurality of inwardly extending channels and the channels may be aligned in first and second directions. For example, five or seven channels may be generally aligned in a first direction and five or seven channels may be generally aligned in a second direction. The channels may be disposed at an angle such as about 90° degrees, but the channels could be disposed at any suitable angle. In addition, one or more depressions may be at least partially disposed within the channels. The inner surface of the lid 58 may also include one or more inwardly extending portions, which may be similar to the inwardly extending portions of the sides 30, 32, 34. The grooves, depressions and/or inwardly extending portions of the lid 58 are preferably integrally formed as part of a unitary, one-piece structure during the blow-molding process.

Therefore, the body 12 of the composter 10 is preferably constructed from one or more blow-molded plastic components. Advantageously, these blow-molded plastic components may be integrally formed as part of a unitary, one-piece structure. In addition, the body 12 of the composter 10 is preferably constructed with an inner wall or surface that is spaced apart from the outer wall or surface, and a hollow interior portion is preferably disposed between the inner wall and the outer wall. The inner wall, outer wall and hollow interior portion are preferably integrally formed as part of the unitary, one-piece structure during the blow-molding process.

The spaced apart walls and hollow interior portion of the blow-molded plastic components may help insulate the composter 10, which may help retain heat within the composter and/or reduce heat loss from the composter. Because the composting of materials is desirably conducted at an elevated temperature, the insulating properties of the spaced apart walls and hollow interior portion may be very advantageous. Thus, the insulating properties of the blow-molded plastic components may facilitate heating of the compost and keeping the compost at a desired temperature. In addition, because the composter 10 may be fully enclosed, it may allow a high temperature to be reached.

For example, active composting generally requires a temperature of about at least 50° F. for most microbe growth and activity. The insulating properties of the composter 10 may help maintain this temperature. In addition, because the composter 10 may be readily portable, the composter may be placed in a garage or shed during the winter in colder climates to help maintain the desired temperature. The composter 10 may also be disposed in other locations if other temperatures are desired. For instance, if a temperature of about 120° F. is desired for compost decomposition, then the composter 10 may be placed outdoors during the summer. The composter 10 may also be placed in direct sunlight, if desired. This may allow the composter 10 to reach temperatures of about 130° F. or more, which may allow unwanted pathogens to be effectively killed. Because the composter 10 may be primarily constructed from blow-molded plastic, it may be able to withstand and be used in a wide variety of environments and temperature ranges. In addition, because the blow-molded plastic may help insulate the composter 10, the desired temperatures may be maintained.

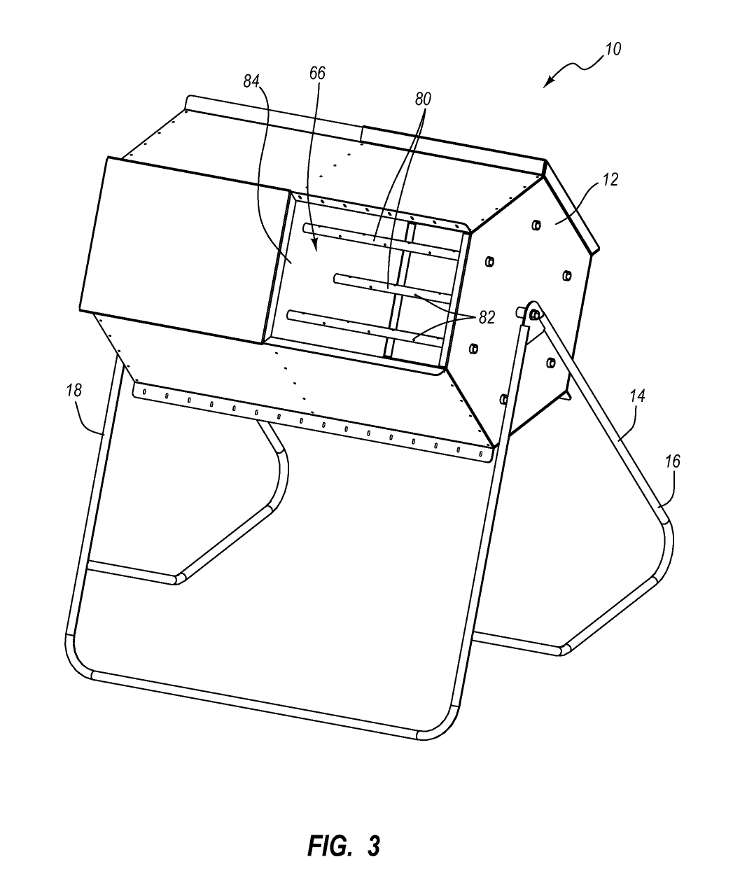

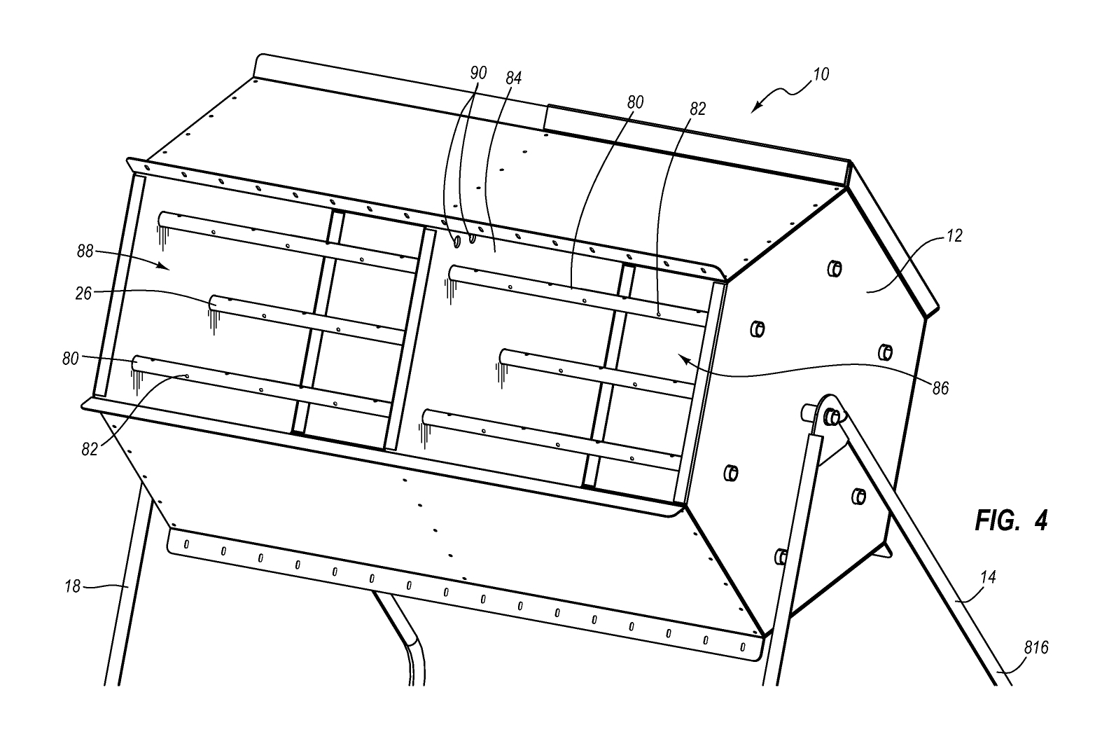

As seen in FIGS. 3 and 4, the composter 10 may have other shapes, sizes, configurations and arrangements. For example, the composter 10 may have a body 12 with six sides and the frame 14 may include first and second support structures 16, 18 with a different configuration. The composter 10 may also include one or more elongated members 80 that are at least partially disposed in the body 12. In particular, the elongated members 80 may be one or more hollow tubes and the elongated members may be disposed within a compartment 66. Significantly, the elongated members 80 may agitate the material with the compartment 66 as the composter 10 is rotated.

The elongated members 80 may also provide increased aeration and more oxygen to the compost during the composting process. For example, the elongated members 80 may extend through the compartment 66 and one or both ends of the elongated members may extend through the exterior wall of the body 12. The elongated members 80 may include one or more openings or passages 82 to facilitate the flow of gas within the composter 10. The elongated members 80 may also allow outside air to be introduced into the compartment 66 of the composter 10. Advantageously, the elongated members 80 may both provide increased aeration and agitate the material when the composter is rotated, which may expedite the decomposition process. If desired, the axle 26 may also be used to increase aeration and agitate the material, which may allow the number of elongated members 80 to be reduced.

As seen in FIGS. 3 and 4, the composter 10 may include a partition 84 and the partition may divide the compartment 66 into one or more sections. For example, as best seen in FIG. 4, the partition 84 may divide the compartment 66 into a first section 86 and a second section 88. It will be appreciated that the composter 10 may include any number of partitions 84 and the compartment 66 may be divided into any number of sections.

If the composter 10 has a plurality of compartments, then the different sections may allow composting of different materials. The different sections may also allow composting at different times and/or amounts. For example, one section may be used to create fresh compost, which may be formed in two to three weeks, and another section may be used to create cured compost, which may be formed in two to three months. Thus, the different sections may allow different materials to be composted, different types of compost to be created, different amounts of compost to be created, different degrees of composting to be completed, etc.

As shown in FIG. 4, the partition 84 may be used to divide the compartment into two sections 86, 88 that are of generally equal size. The partition 84, however, could divide the compartment 66 into differently sized sections. The partition 84 could be movable to allow the size of the sections to be adjusted. For example, the partition 84 may slide between various locations to allow the size of the sections 86, 88 to be increased or decreased. The partition 84 may also be slidable, for example, to allow the size of the first section 86 to be increased and the size of the second section 88 to be decreased. Thus, the partition 84 may be moved into various positions to change the relative sizes of the sections.

The partition 84 may be slidable relative to the axle 26 and/or the elongated members 80. In particular, the partition 84 may include openings and the axle 26 and/or the elongated members 80 may be disposed within the openings. When it is desired to move the partition 84, it may be slid along the axle 26 and/or elongated members 80. Preferably, the partition 84 may move the entire width of the compartment 66 to form sections 86, 88 of various sizes, but the range of movement of the partition may be limited or fixed. In addition, it will be appreciated that the partition 84, elongated members 80 and axle 26 may not be required.

In order to facilitate movement of the partition 84, the outer edges or perimeter of the partition may be disposed in one or more receiving portions formed in the body 12. The partition 84 could also be movable along a track, on rollers, and the like to facilitate movement of the partition. The partition 84 may also be deformed or bent to facilitate movement of the partition. For example, the partition may be constructed from a flexible material, such as fabric, and the partition may be deformed to facilitate movement.

Advantageously, the movable partition 84 may allow the size of the sections 86, 88 within the compartment 66 of the composter 10 to be readily changed. For example, when the material in a first section 86 decomposes, it may reduce in size. If desired, the position of the partition 84 may be adjusted because of the decreasing size of the decomposing materials. This may increase the size of the second section 88 and permit additional material to be added to the second section.

The partition 84 may completely separate the sections 86, 88, or it may allow communication between the sections. For example, the partition 84 may permit fluids, such as leachate, to pass between the sections 86, 88. Thus, the partition 84 may include one or more openings 90 to allow fluid transfer between the sections 86, 88. The openings 90 may be disposed proximate an outer portion of the partition 84 and the openings may be sized and configured to help control the amount of fluid passing between the sections. The partition 84 could also include one or more permeable sections that are sized and configured to allow fluid to pass between the compartments 86, 88. For instance, the partition 84 may be constructed from permeable materials or sections such as screens, mesh, and the like. Because the partition 84 may permit fluids to pass between the sections 86, 88, this may allow drier materials in one section to absorb fluids from the other section. This may, for example, eliminate the need to add water, moisture or other materials to the section with the drier materials. This may also eliminate the need to remove water, moisture and/or leachate from the other section. This may expedite the composting process and/or make composting quicker and easier. In addition, because the partition 84 may permit fluids to pass between the sections 86, 88, this may allow the fluids to be evenly distributed within the compartment 66. The partition 84 may be constructed from various materials such as metal (such as steel or aluminum), plastic (such as vinyl or nylon), fabric (such as cloth or polyester), or other suitable natural or synthetic materials depending, for example, upon the intended purpose of the partition.

If desired, the partition 84 that may be disposed at an angle relative to the body 12. For example, the partition 84 may be disposed perpendicular to an inner wall of the body 12. The partition 84 may also be disposed at other angles relative to the body 12, such as about 15°, 30°, 45° or 60° angles, and the like. The partition 84 may be pivotally connected to the body 12 and it may be secured in one or more desired positions. The partition 84 may also be movable relative to an axis of rotation of the body 12. For example, if the body 12 rotates relative to a generally horizontal axis, then the partition 84 may be movable relative to that axis. This may allow the partition 84 to be disposed generally perpendicular to the axis, generally aligned with the axis, spaced apart from the axis and/or generally parallel to the axis. The partition 84 may be disposed at any suitable angle relative to the axis such as about 15°, 30°, 45°, 60° angles, and the like.

In greater detail, the body 12 of the composter 10 may rotate about the axle 26 and the partition 84 may be disposed at an angle relative to this axis of rotation. As shown in FIG. 4, the partition 84 may be disposed perpendicular to the axle 26 and the axis of rotation of the composter 10. The partition 84, however, could be disposed at other suitable angles relative to the axle 26 and/or the axis of rotation.

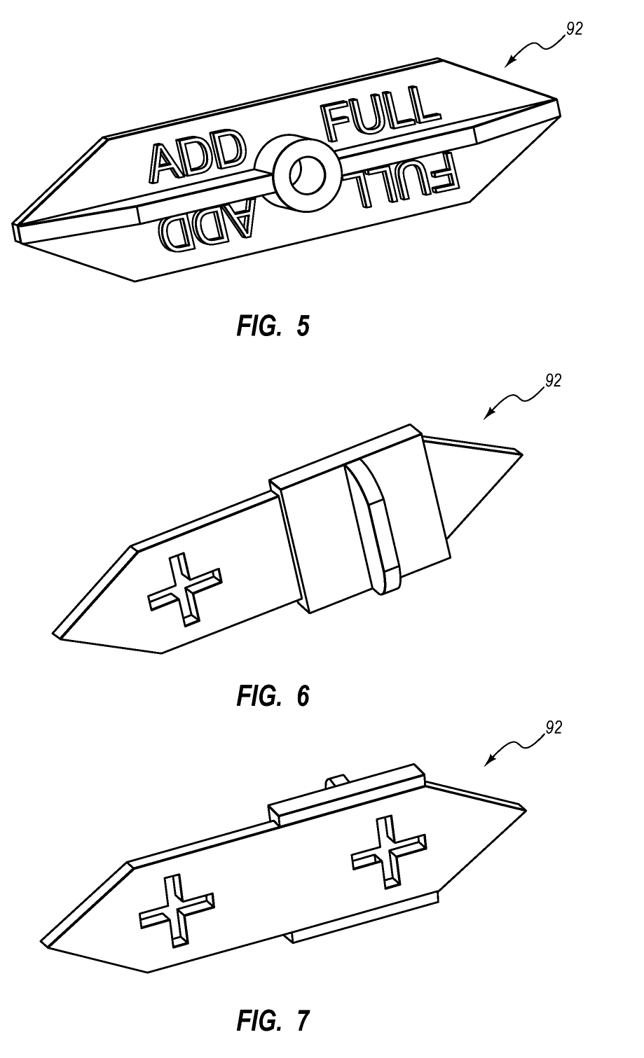

As shown in FIGS. 5-7, the composter 10 that may include one or more indicators 92. The indicators 92 may selectively indicate which, if any, sections 86, 88 should receive additional materials. The indicators 92 may indicate, for example, that the first section 86 is in the accumulation stage and more composting materials should be added. The indicators 92 may also indicate that the second section 88 is in the composting stage and no additional material should be added. In addition, the indicators 92 may indicate that both the first and second sections 86, 88 are in the accumulation stage or composting stage. The indicators 92 may also provide other information, if desired. It will be appreciated that the indicators 92 may have other suitable shapes, sizes, configurations and arrangements, and the indicators may also be in the form of pointers, sliders, dials and the like.

As mentioned above, the composter 10 is preferably at least partially constructed from lightweight materials such as blow-molded plastic. Advantageously, the lightweight materials may help reduce the weight of the composter 10, which may facilitate shipping, transportation and movement of the composter. This may also allow the composter to be quickly and easily moved between a garage, backyard, patio, shed and the like.

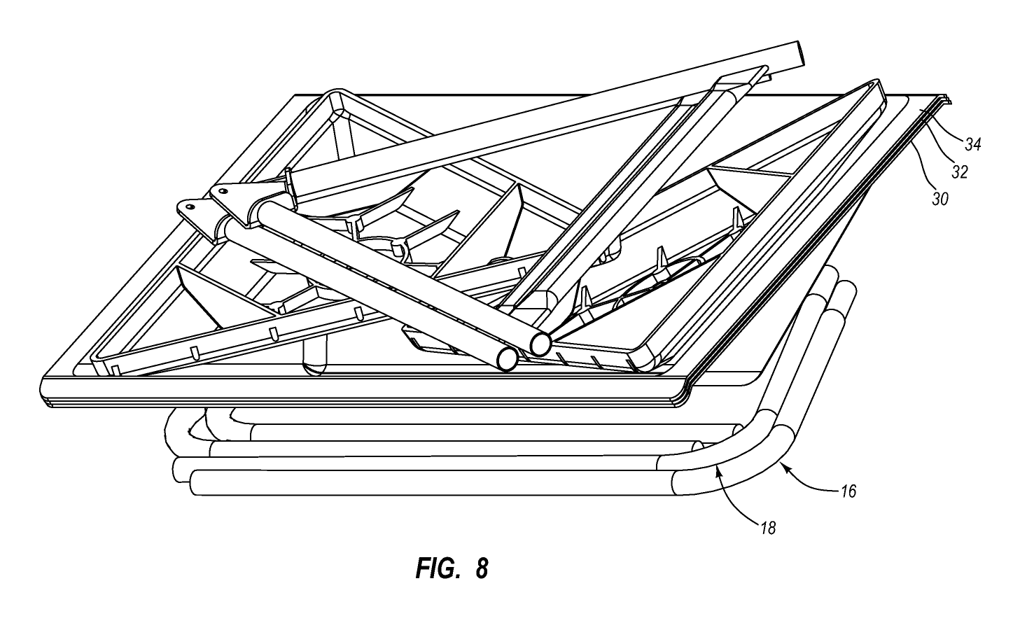

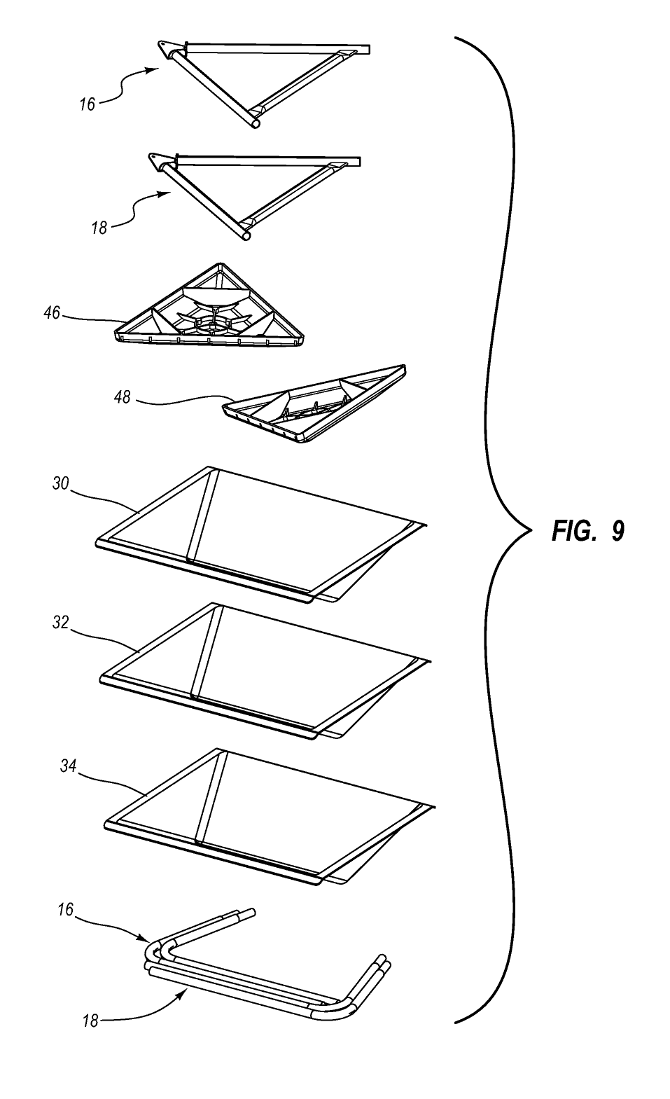

The composter 10 may also be readily assembled and/or disassembled. For example, the composter 10 may be disposed in a first configuration for shipping, storage and/or transport and a second configuration for use. In particular, in the shipping configurations as shown in FIGS. 8 and 9, the sides 30, 32, 34 may be stacked and nested together. In addition, the first and second support structures 16, 18 of the frame 14 may be constructed from one or more components and at least some of these components may be disposed within one of the sides 30, 32, 34. Because some of the components may be stacked and nested together, and other components may be disposed within the nested components, that may reduce the size of the packaging. Advantageously, the smaller packaging may reduce the amount of storage space and may help reduce shipping costs.

In use, the composter 10 may be filled with a desired amount of composting materials and ingredients, such as leaves, yard trimmings and household waste. The body 12 of the composter 10 may be simply rotated about its axle 26, which may be horizontally disposed, to mix and aerate the compost. Because the body 12 of the composter 10 may be disposed above the ground, it may allow materials to be quickly and easily added and removed from the composter. This may also allow the mixture of materials in the composter 10 to be readily changed. For example, if a particular carbon-to-nitrogen ratio is desired, then the composter 10 may allow materials with high carbon content and/or high nitrogen content to be added or removed. Thus, the composter 10 may allow a proper ratio of materials to be maintained and balanced.

Although this invention has been described in terms of certain preferred embodiments, other embodiments apparent to those of ordinary skill in the art are also within the scope of this invention. Accordingly, the scope of the invention is intended to be defined only by the claims which follow.

What is claimed is:

1. A composter comprising:
a body including a compartment that is sized and configured to receive composting materials, the body comprising:
a first portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction;
a second portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction; and
a third portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction; and
a frame connected to the body, the frame being sized and configured to support the body above a support surface.

2. The composter as in claim 1, wherein the outer wall of the first portion of the body includes an inwardly curved, generally concave portion that is disposed between a first outwardly curved, generally convex portion and a second outwardly curved, generally convex portion.

3. The composter as in claim 1, further comprising a handle integrally formed in the outer wall of the first portion of the body as part of the unitary, one-piece construction.

4. The composter as in claim 1, wherein the first portion forms a first side of the body, the second portion forms a second side of the body and the third portion forms a third side of the body; and
wherein the compartment is at least partially defined by the first portion, the second portion and the third portion.

5. The composter as in claim 1, further comprising a lid attached to the first portion of the body, the lid covering an opening to the compartment, the lid including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction.

6. The composter as in claim 1, further comprising a front portion connected to the first portion, the second portion and the third portion of the body, the front portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction; and
further comprising a rear portion connected to the first portion, the second portion and the third portion of the body, the rear portion including an inner wall, an outer wall and a hollow interior portion disposed between the inner wall and the outer wall, the inner wall including a plurality of inwardly extending channels, the inner wall, the outer wall, the hollow interior portion and the channels being integrally formed as part of a unitary, one-piece construction.

7. The composter as in claim 6, further comprising an axle extending through the front portion and the rear portion of the body, the axle being rotatably connected to the frame.

8. The composter as in claim 6, wherein at least some of the channels in the front portion are aligned with a central portion of the front portion and extend radially outwardly.

9. The composter as in claim 6, further comprising one or more depressions at least partially disposed in at least some of the channels in the front portion of the body.

10. The composter as in claim 6, wherein the channels in the front portion of the body are disposed in a generally symmetric pattern that covers at least a majority of the inner wall of the front portion of the body.

11. The composter as in claim 1, further comprising a partition disposed within the compartment, the partition dividing the compartment into a first section and a second section.

12. The composter as in claim 11, wherein the partition is sized and configured to allow fluid communication between the first section and the second section of the compartment.

13. The composter as in claim 1, further comprising one or more elongated gas flow members disposed in the compartment, the elongated members being sized and configured to help mix the composting materials when the body is rotated and allow gas flow within the compartment.

14. The composter as in claim 1, further comprising an inwardly extending portion on the inner wall of the first portion of the body, the inwardly extending portion being integrally formed as part of the unitary, one-piece construction.

15. The composter as in claim 14, wherein the inwardly extending portion has a generally X-shaped configuration and is sized and configured to facilitate mixing of the composting materials in the body.

16. The composter as in claim 14, wherein the inwardly extending portion has a height generally equal to or greater than an average depth of the channels in the inner wall of the first portion of the body.

17. The composter as in claim 1, wherein the plurality of inwardly extending channels in the inner wall of the first portion of the body includes at least five generally aligned channels.

18. The composter as in claim 1, wherein the plurality of inwardly extending channels in the inner wall of the first portion of the body includes a first set of generally aligned channels that are spaced apart from a second set of generally aligned channels.

19. The composter as in claim 1, wherein the plurality of inwardly extending channels in the inner wall of the first portion of the body includes a first set of generally aligned channels that are disposed generally perpendicular to a second set of generally aligned channels.

20. The composter as in claim 1, further comprising at least one depression disposed in at least a majority of the inwardly extending channels in the inner wall of the first portion of the body.
